(19) United States
(12) Patent Application Publication
(10) Pub. No.: US 2026/0355478 A1
(43) Pub. Date: Nov. 12, 2026

(54) IMPLANTABLE DEVICE TO ENSURE STOMA PATENCY

(71) Applicant: MASSACHUSETTS INSTITUTE OF TECHNOLOGY, Cambridge, MA (US)

(72) Inventors: Gabriel FRIEDMAN, Cambridge, MA (US); Ellen ROCHE, Cambridge, MA (US); Luca ROSALIA, Cambridge, MA (US); Jonas SOGBADJI, Cambridge, MA (US); Bryce STARR, Cambridge, MA (US)

(21) Appl. No.: 19/475,060

(22) PCT Filed: Apr. 17, 2024
(86) PCT No.: PCT/US2024/024883

§ 371 Date: Oct. 14, 2025

Related U.S. Application Data
(60) Provisional application No. 63/496,628, filed on Apr. 17, 2023.

(51) Int. Cl. A61M 27/00; A61F 2/90; A61F 2/95

(52) U.S. Cl. CPC A61M 27/006; A61F 2/90; A61F 2/95

(57) ABSTRACT

An improved hydrocephalus stent has been developed which exhibits greater stability at the site of implantation and less occlusion. The expandable hydrocephalus stent device includes a first side, a middle and a second side, wherein the stent device is compressible into an endoscopic delivery catheter, wherein the stent device expands after release from the delivery catheter between the prepontine cistern and the third ventricle, the first side expanding within the prepontine cistern and the second side expanding within the third ventricle. The stent device includes a central channel to funnel fluid from the third ventricle into the prepontine cistern and has a curved surface between the first and the second sides of the central channel to secure the device to the membrane Prepontine Cistern between the prepontine cistern and the third ventricle.

### CROSS-REFERENCE TO RELATED APPLICATIONS

This application claims the benefit of and priority to U.S.S.N. 63/496,628, filed on Apr. 17, 2023, which is incorporated by reference herein in its entirety.

### STATEMENT REGARDING FEDERAL SPONSORED RESEARCH

This invention was made with NO government support.

### BACKGROUND OF THE INVENTION

Hydrocephalus is a condition with an average incidence of 88 in 100,000 pediatrics patients and caused by an accumulation of excess cerebrospinal fluid (CSF) within the brain that commonly occurs idiopathically, via a surplus of CSF production or absence of CSF reabsorption (Isaacs, et al. (2017). Age-specific global epidemiology of hydrocephalus: Systematic review, metanalysis and global birth surveillance. PLoS ONE, 13(10). https://doi.org/10.1371/journal.pone.0204926). This results in an increased intracranial pressure (ICP), widening of the third ventricles, and subsequently can cause a range of symptoms including headaches, seizures, or mental impairment. Without surgical treatment, death or severe disability may occur.

An endoscopic third ventriculostomy (ETV) is an endoscopic surgical procedure which creates a lumen in the translucent tuber cinereum at the base of the third ventricle to allow excess CSF to exit the ventricular system into the basal cisterns. This allows CSF to be reabsorbed naturally through the arachnoid granulations, providing treatment of hydrocephalus. Of the completed ETV procedures, 20-50% are complicated by closure of the surgically created stoma, requiring either an additional ETV procedure or placement of a ventriculo-peritoneal shunt (VPS), which carries a significantly higher complication profile (Walker, M. (2021, Jan. 24). ETV Complications. Retrieved Feb. 7, 2023, from https://www.hydroassoc.org/etv-complications/#:~:text=Sudden%20closure%20of%20the%20pathway, six%20months%20of%20the%20operation.).

The ETV procedure allows for a patient to reabsorb CSF naturally without hardware that traverses from the brain to the abdomen, as the VPS procedure does, thereby lowering the infection risk for the ETV procedure. The UCLA neurosurgery department estimates that there are 33,000 VPS placed per year—about one every 15 minutes—in the United States and that each shunt placement will require, on average, between 3 and 5 additional surgeries throughout the lifetime to maintain their integrity. This represents the scope and burden that the ETV and VPS procedures placing on the hydrocephalus patient population.

Currently, 20-50% of ETV procedures are complicated by closure (loss of patency [the status of an unobstructed lumen]) of the surgically-created opening in the floor of the 3rd ventricle (stoma) and a large percentage of these complications arise in the first 6 months after operation. (Walker, M. (2021, Jan. 24). ETV Complications. Retrieved Feb. 7, 2023, from https://www.hydroassoc.org/etv-complications/#:~:text=Sudden%20closure%20of%20the%20pathway, six%20months%20of%20the%20operation). To date, there are no devices or methods available to the public that provide lasting stomal patency in patients who receive the ETV procedure. The ETV procedure is a strong operative solution for cases of obstructive hydrocephalus and as an alternative to shunt revision. Data show that ETV is associated with potentially lower complication rates (particularly infection) and costs than VPS. Further, unlike VPS, ETV does not require MRI surveillance and does not relying on communicating hardware to the abdomen. However, the success of ETV is currently hindered by elevated stoma closure rates (Lu, L. et al. (2019, September). Endoscopic Third Ventriculostomy versus Ventriculoperitoneal Shunt in Patients with Obstructive Hydrocephalus:

Meta-Analysis of Randomized Controlled Trials. World Neurosurgery, 129. https://doi.org/10.1016/j.wneu.2019.04.255).

The current alternative, the VPS, has a high infection risk from communication with the abdominal cavity. The VPS hardware can become occluded leading to return of hydrocephalus symptoms. The VPS requires, on average, 3-5 additional operations per shunt placed to manage complications (UCLA Health, What is hydrocephalus? Retrieved Apr. 15, 2023. https://www.uclahealth.org/medical-services/pediatric-neurosurgery/conditions-treatment/pediatric-hydrocephalus-program/hydrocephalus-faqs#:~:text=Hydrocephalus%20occurs%20in%20two%20out, annually%20in%20the%20United%20States). The ETV procedure with the stoma patency device directly aims to prevent occlusion and does not rely on any communicating hardware that leaves the skull.

Given the infection risk for VPS and the established failure rates for ETV, there is a well-defined clinical need for an improved and durable treatment of hydrocephalus (Walker, M. (2021, Jan. 24). ETV Complications. Retrieved Feb. 7, 2023, from https://www.hydroassoc.org/etv-complications/#:~:text=Sudden%20closure%20of%20the%20pathway, six%20months%20of%20the%20operation.).

In addition to the clinical need for an improved ETV procedure, there is a clinical need to evaluate stoma (the surgically created hole in the ETV procedure) patency (the status of remaining open) for patients after receiving ETV. Clinically, the standard of care for evaluation of CSF flow in VPS is MRI imaging, which suffers from several limitations, including high costs and the lack of spatio-temporal resolution to detect accurate CSF flow within a cardiac cycle. Further, current literature is inconsistent on the significance of flow interpretation and treatment planning (Bradley, W G J et al. (1996). Normal-pressure hydrocephalus: evaluation with cerebrospinal fluid flow measurements at MR imaging. Radiology, 198(2). 10.1148/radiology.198.2.8596861 and Luetmer, P H et al. (2002). Measurement of cerebrospinal fluid flow at the cerebral aqueduct by use of phase-contrast magnetic resonance imaging: technique validation and utility in diagnosing idiopathic normal pressure hydrocephalus. Neurosurgery, 50(3). 10.1097/00006123-200203000-00020).

It is therefore an object of the present invention to create technology that optimizes the procedure so that it can become the new standard of care for eligible patients owing to its 1) improved success rate, 2) decreased infection risk, 3) absence of implanted hardware that travels from the brain to the abdomen as in the VPS procedure, and 4) high rates of usage in pediatric patients.

It is a further object of the invention to decrease the rates of return operations and complications in this vulnerable patient group.

It is another object of the present invention to provide both therapeutic relief from hydrocephalus symptoms as well as to offer a diagnostic assay to monitor the CSF micro dynamics within the ventricle.

It is another object of the present invention to reduce healthcare spending on hydrocephalus management as is currently the case with VPS management.

### SUMMARY OF THE INVENTION

An improved hydrocephalus stent has been developed which exhibits greater stability at the site of implantation and less occlusion. The expandable hydrocephalus stent device includes a first side, a middle and a second side, wherein the stent device is compressible into an endoscopic delivery catheter, wherein the stent device expands radially and compressively after release from the delivery catheter between the prepontine cistern and the third ventricle; the first side expanding within the prepontine cistern and the second side expanding within the third ventricle. The stent device includes a central channel to funnel fluid from the third ventricle into the prepontine cistern and has a curved surface between the first and the second sides of the central channel to secure the device to the membrane between the prepontine cistern and the third ventricle. In one embodiment, the expandable hydrocephalus stent device of claim 1 includes a slidable wire or filament to adjust the diameter of the central channel of the stent device to conform to the membrane after insertion. In another embodiment the expandable hydrocephalus stent device includes an impenetrable membrane channel between the first and the second sides of the stent device to reduce or prevent cell growth that could occlude the central channel of the stent device. In another embodiment, the stent may contain central connectors to increase the radial strength of the device and enable deployment of the device in a controlled manner. In one embodiment, the device contains eyelet holes at the tips of the leaflets. First, these holes would serve as connection points to suture threads which can be pulled through the deployment tool for device loading. Second, they would increase the radio-opacity of the device, allowing for better visualization under radiation imaging.

As shown in the figures, the expandable hydrocephalus stent device typically is circular, but may also be star shaped. In another embodiment, the first and/or second sides of the stent device include “hooks” or “clamps” that are used to secure the stent device to the membrane.

The method of inserting the hydrocephalus stent device is stepwise. Initially, the ETV procedure creates the stoma described previously. Deployment is sequential and conducted under direct visualization via the wide-angle endoscopic camera. First, the endoscope is advanced with the stoma between the third ventricle and prepontine cistern in view. The catheter is advanced within the endoscope enabling the device to protrude to its half-deployed state within the prepontine cistern. Reverse traction is placed on the endoscope to bring the distal disk in contact with the prepontine side of the membrane. Ensuring the membrane is in contact with the deployed disk is a mechanism to confirm proper positioning and validates that the impermeable component of the device spans the ventricular membrane to keep the stoma patent. The endoscope is then retracted while the last disk is deployed. Radial expansion and compression of the device secures the membrane between the disks and keeps the stoma patent to allow CSF to drain. In one embodiment the diameter of the stent device is then adjusted; in another the stent device is secured by hooks or clamps to the membrane.

### BRIEF DESCRIPTION OF THE DRAWINGS

FIG. 1 is a cross-sectional view of the device in its undeployed configuration within a catheter before insertion into the lumen created by an endoscopic third ventriculostomy.

FIG. 2 is a cross-sectional view of the first half of the device as it is deployed into the prepontine cistern, while the smaller middle channel, bound by an impermeable membrane, sits in the ventricular membrane between the third ventricle and the prepontine cistern.

FIG. 3 is a cross-sectional view of the device fully released from the catheter, where the second half of the device is deployed into the third ventricle, effectively sandwiching the membrane of the translucent tuber cinereum.

FIG. 4 is a cross-sectional view of the device showing adjusting the size of the device to ensure a tight fit with the membrane wall, in which a pre-attached wire is pulled, causing the implanted device's height to shrink to the thickness of the membrane.

FIG. 5 is a side view of the expanding hydrocephalus stent.

FIG. 6 is a top view of the expanding hydrocephalus stent.

FIG. 7 is a side view of an expanding hydrocephalus stent showing a channel to prevent cell growth.

FIG. 8 is a top view of an expanding hydrocephalus stent demonstrating what the expanding hydrocephalus stent appears as with both a channel membrane that prevents cell growth and curved faces that cause an interference fit between the device and the membrane.

FIG. 9 is a side view of hooks attached to the channel circumference.

FIG. 10 is a top view of the expanding hydrocephalus stent, showing how both an impermeable membrane and wire are attached. The impermeable membrane ensures the lumen remains open and free of cell growth, while the wire allows for tightening of the device to create a better fit between the membrane of the translucent tuber cinereum and the device.

FIGS. 11A and 11B are cross-sectional views of the expanding hydrocephalus stent with central connectors to increase the radial strength of the device as well as the eyelet holes at the tips of the leaflets, serving as attachment points for the sutures of the device to be pulled during loading into the delivery tool.

### DETAILED DESCRIPTION OF THE INVENTION

### Expandable Hydrocephalus Stent

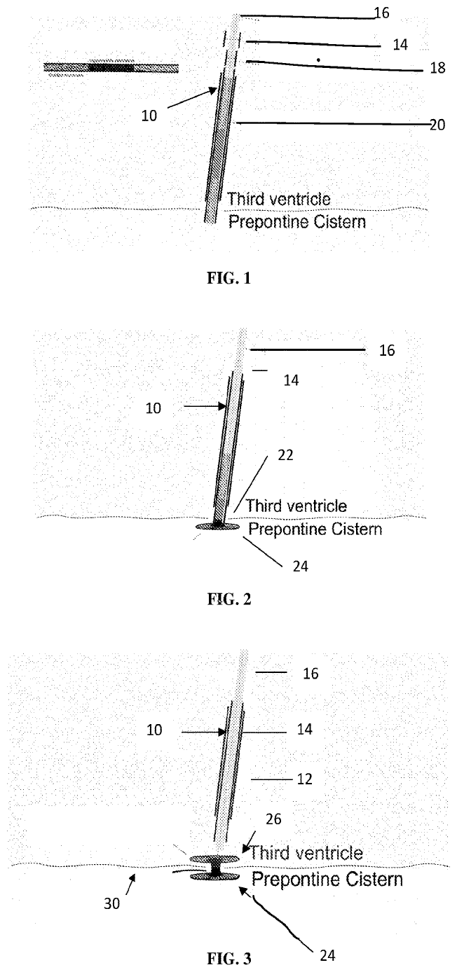

Embodiments of the expanding hydrocephalus stent are shown in FIGS. 1-11.

As shown in FIG. 1, the undeployed device 20 sits in its undeployed configuration within an endoscope channel 18 where it is inserted using stent delivery catheter 16(1) before insertion into the lumen created by the endoscope 14, forming an endoscopic third ventriculostomy in the prepontine cistern. Then, the device 20 is deployed incrementally through 14. Referring to FIG. 2, the first half of the device 22 is deployed though an endoscope 14 into the prepontine cistern, while the smaller middle channel 24, bound by an impermeable cell membrane, sits in the membrane between the third ventricle and the prepontine cistern. FIG. 3 shows device 22 fully released from the catheter, and the second half 26 of the device is deployed into the third ventricle, effectively sandwiching the membrane of the translucent tuber cinereum.

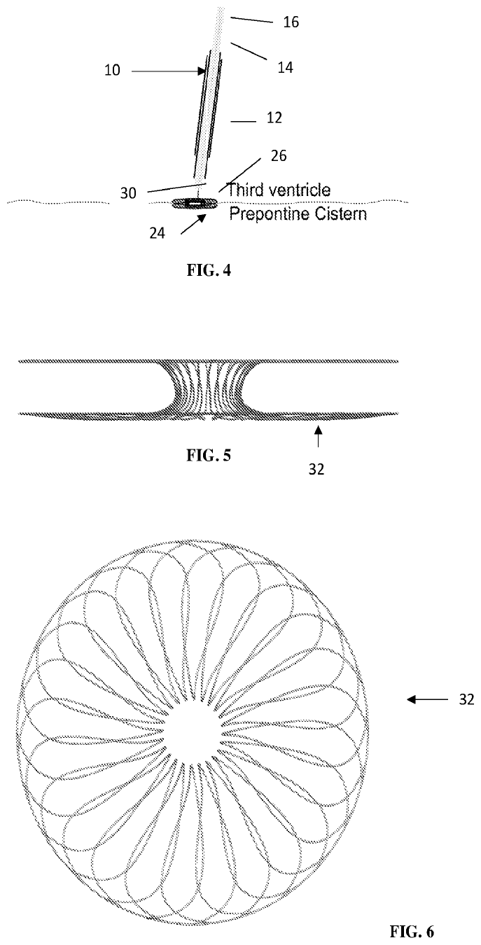

FIG. 4 shows a method for adjusting the size of the implanted device 32 to ensure a tight fit with the membrane wall, in which a pre-attached wire 30 is pulled, causing the implanted device's height to shrink to the thickness of the membrane.

The middle segment of smaller radius is preferably bound circumferentially by a material impermeable to cell proliferation to prevent luminal obstruction, scarring or stenosis, but this is not essential.

The device could alternatively be equipped with a small wire through the catheter that, when pulled, would tighten the fit between the device and the membrane by shortening the distance between the two halves and clamping the membrane of the translucent tuber cinereum between them, as seen in FIG. 4.

FIG. 5 and FIG. 6 display a basic example of the expanding hydrocephalus stent device 32.

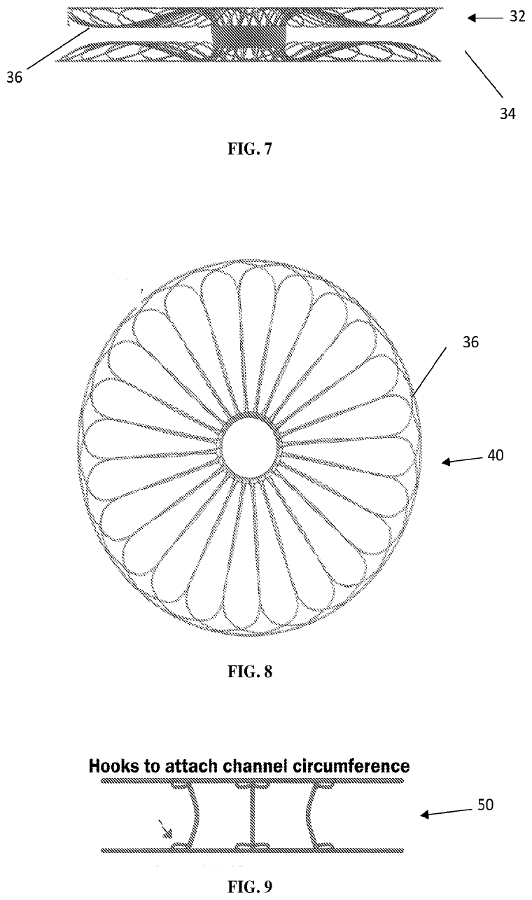

In some embodiments, it is desirable for the two expanded ends of the device to create a press fit with the thin membrane of the brain clamped between them to ensure the device remains in place, see FIG. 7.

FIG. 7 (side view) and FIG. 8 show the expanding hydrocephalus stent 32 with a channel 34 lined with a channel membrane that prevents cell growth and curved faces 36a, 36b that secure the membrane in place, thereby preventing it from dislodging along the device. Curved faces 36a, 36b also help reduce the stress of the stent 32 on the tissue, minimizing the risk of trauma during and following implantation. The device should not need to withstand much force, flow, or turbulence, and the membrane of the brain is on average 3 mm thick, so an interference fit with the device is possible if the two extended ends of the device have less than 3 mm between them.

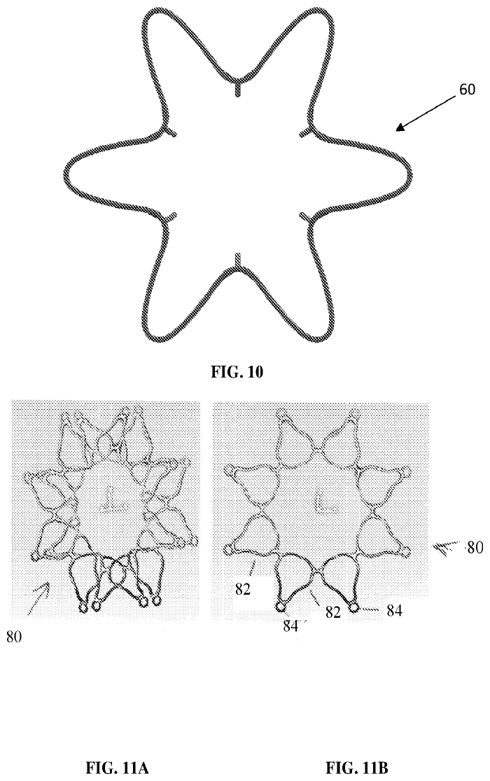

FIG. 9 and FIG. 10 illustrate two additional prototypes, including both an impermeable membrane and wire (shown in FIG. 4, not shown here) to be attached. The impermeable membrane ensures the lumen remains open and free of cell growth, while the wire allows for the tightening of the device to create a better fit between the membrane of the translucent tuber cinereum and the device. In FIG. 9, the hooks are used as anchoring points between the membrane and the channel. The device shown in FIG. 10 has a six-arm star-like profile. The device features hooks for attachment of the membrane to the channel. All prototypes have the same purpose and serve the same application.

FIGS. 11A and 11B illustrate an embodiment 80 with central connectors 82 and eyelet holes 84. The central connectors 82 provide additional radial strength compared to other designs, and the eyelet holes 84 provide suture attachment points for loading of the device 80 into a delivery tool.

The device is preferably made of a material such as Nitinol, a biocompatible metal alloy of nickel and titanium with unique properties, including superelasticity or pseudoelasticity and “shape memory.” It also provides radio-opaque properties to the completed product, enabling imaging under fluoroscopy. Preferably the device would be of a biocompatible nitinol material, with the design cut from a nitinol tube in the undeployed configuration (see FIG. 1) and heat set to the deployed configuration (see FIG. 3) with the arms extending out radially. The temperature at which Nitinol (nickel-titanium alloy) goes back to its pre-deformed shape (here referred to as the Austenite Finish Temperature, Af) can range from 15 to 40 degrees Celsius. However, a design made directly from nitinol wire or an alternative biocompatible, braided textile wire would also function similarly.

Alternatively, a device of similar design with a textile-wire component may be coated with a bio-inert, radio-opaque material including, but not limited to, barium sulfate, bismuth, and tungsten. The membrane would be made of a biocompatible, non-porous, hydrophobic, cheap, and widely available material such as Silicone to prevent tissue ingrowth, ensuring long-term patency of the lumen. The thickness of the membrane may range between 0.3 and 1 mm.

### Method of Implanting

The device initially fits inside a low-profile catheter for implantation through a working channel in an endoscope. The catheter constrains the device but when the device is pushed out of the catheter, the device expands into its final deployed stent configuration, due to shape memory and heat setting properties. Both ends of the stent expand significantly radially while shrinking vertically, creating a middle segment that creates an open channel between the floor of the third ventricle and the prepontine cistern for CSF to drain via an established pressure differential.

A stoma patency kit that can be included in the procedure cart for the ETV procedures: an endoscopic catheter (fit to the standard ETV endoscopes), the patency device itself, and a deployment method.

Advantages of the device over existing methods include decreasing the incidence and/or severity of stoma closure complications and reducing infection risk by avoiding VPS hardware.

Additionally, this device would be the first of its kind to serve a diagnostic role for monitoring stoma patency. This could drastically shift the standard of care for longitudinal hydrocephalus care where patients would complete fluoroscopic studies, mere fractions of the cost of MRI, to monitor stoma patency and prevent recurrence of hydrocephalus symptoms.

The commercial applications for this technology within the neurosurgery space alone are immense. From the Hydrocephalus Association, it is anticipated that 36,000 hydrocephalus surgeries (one every 15 minutes) are performed every year. This represents the scope and burden that the ETV and VPS procedures place on the hydrocephalus patient population. With a defined clinical need and well-constructed intervention, the ETV procedure with stoma patency could become an even larger aspect in the hydrocephalus management realm than both the VPS and ETV stand-alone options.

Those skilled in the art will recognize or be able to ascertain using no more than routine experimentation, many equivalents to the specific embodiments of the method and compositions described herein. Such equivalents are intended to be encompassed by the following claims.

1. An expandable hydrocephalus stent device comprising a first side, a middle and a second side, wherein the stent device is compressible into an endoscopic delivery catheter, wherein the stent device expands after release from the delivery catheter between the prepontine cistern and the third ventricle, the first side expanding within the prepontine cistern and the second side expanding within the third ventricle, the stent device comprising a central channel to funnel fluid from the third ventricle into the prepontine cistern, and having a curved surface between the first and the second sides of the central channel to secure the device to the membrane between the prepontine cistern and the third ventricle.

2. The expandable hydrocephalus stent device of claim 1 comprising a slidable wire or filament to adjust the diameter of the central channel of the stent device to conform to the membrane after insertion.

3. The expandable hydrocephalus stent device of claim 1 comprising a channel between the first and the second sides of the stent device to reduce or prevent cell growth that could occlude the central channel of the stent device.

4. The expandable hydrocephalus stent device of claim 1 wherein the outer perimeter of the stent device is circular.

5. The expandable hydrocephalus stent device of claim 1 wherein the outer perimeter of the stent device is star shaped.

6. The expandable hydrocephalus stent device of claim 1 wherein the stent device comprises hooks or clamps to secure the stent device to the membrane.

7. The expandable hydrocephalus stent device of claim 1 wherein connectors are included in the central part of the device to increase its radial strength.

8. The expandable hydrocephalus stent device of claim 1 wherein the leaflets of the device include eyelet holes as attachment points to suture threads to pull the device into the deployment tool during loading.

9. The expandable hydrocephalus stent device of claim 1 wherein the leaflets of the device include eyelet holes that can further increase the radio-opacity of the device for visualization under radiation imaging or which can be used to secure the device when implanted.

10. The expandable hydrocephalus stent device of claim 1 formed of a metal alone like stainless steel or formed of a metal alloy like Nickel-Titanium, or formed of silicone, or formed of or coated with a radiopaque material, or formed of and/or coated with an anti-cell proliferation agent.

11. A method of inserting the hydrocephalus stent device of claim 1 comprising:
inserting an endoscope containing a stent delivery catheter having therein the stent device of claim 1 into an individual to structurally support an opening in the membrane between the third ventricle and the prepontine cistern, and
inserting the stent device into the opening, wherein the first side of the stent device is in the prepontine cistern, the middle component of the device abuts the edges of the ventricular membrane, and the second side of the stent device is in the third ventricle.

12. The method of claim 10 further comprising adjusting the diameter of the stent device.

13. The method of claim 10 further comprising adjusting the number of leaflets of the stent device.

14. The method of claim 10 further comprising adjusting the number and thickness of connectors through the central part of the device to provide adequate radial strength of the device.

15. The method of claim 10 further comprising adjusting the number and size of eyelet holes at the leaflets.

16. The method of claim 10 further comprising adjusting the distance between the first and second sides of the device.

17. The method of claim 10 further comprising adjusting multiple materials described in the fabrication of the device.

18. The method of claim 10 further comprising adjusting the transition temperature of any shape-memory allow described in the fabrication of the device.

19. The method of claim 10 further comprising securing the stent device to the membrane.
